(19) United States
(12) Patent Application Publication
(10) Pub. No.: US 2012/0095339 A1
(43) Pub. Date: Apr. 19, 2012

(54) ULTRASOUND DIAGNOSTIC APPARATUS

(75) Inventor: Rika TASHIRO, Ashigara-kami-gun (JP)

(73) Assignee: FUJIFILM CORPORATION, Tokyo (JP)

(21) Appl. No.: 13/271,344

(22) Filed: Oct. 12, 2011

(30) Foreign Application Priority Data
Oct. 13, 2010 (JP) 2010-230784

(51) Int. Cl. A61B 8/14

(52) U.S. Cl. 600/443

(57) ABSTRACT

An ultrasound diagnostic apparatus includes an ultrasound probe, an image producer, a monitor, a position designating unit for designating a target position when paracentesis is performed, an insertion position calculating unit for calculating an insertion position from information on the target position designated by the position designating unit and a predetermined insertion angle and an insertion position display unit for displaying the insertion position calculated by the insertion position calculating unit. The ultrasound diagnostic apparatus ensures that a puncture needle inserted from a position away from an ultrasound probe to reduce the insertion angle so as to be imaged at a higher definition can also reach a target site.

### BACKGROUND OF THE INVENTION

The present invention relates to an ultrasound diagnostic apparatus and more specifically to an ultrasound diagnostic apparatus that may be used in paracentesis.

Heretofore, ultrasound diagnostic apparatuses that use ultrasound images have been put to use in the medical field. In general, this type of ultrasound diagnostic apparatus includes an ultrasound probe equipped with a built-in oscillator array and an apparatus body connected to the ultrasound probe. The ultrasound probe transmits ultrasonic waves toward a subject and receives ultrasonic echoes from the subject. The received signals are then electrically processed in the apparatus body to generate an ultrasound image.

The ultrasound diagnostic apparatus is also used in paracentesis in which a doctor inserts a puncture device such as a puncture needle to a desired site and collects a tissue sample to make a cell/tissue diagnosis.

In order-to ensure that the puncture needle reaches a target object or site in paracentesis, the doctor inserts the puncture needle into a subject along a predetermined insertion path while seeing an ultrasound image.

It is important in such paracentesis that the puncture needle can be confirmed on a monitor (ultrasound image) and that the puncture needle can be made to reach the target object or site.

To this end, JP 2010-115246 A describes forming a groove for guiding a puncture needle on a lateral side of an ultrasound probe to stably insert the puncture needle according to an insertion path, and vertically providing a plurality of laser light sources on the lateral side of the ultrasound probe to determine the orientation of the puncture needle based on laser beams from the laser light sources so that the puncture needle may be displayed on an ultrasound image corresponding to the ultrasound irradiation surface (scan surface).

JP 2000-166918 A describes interposing a polymer gel between a subject and an ultrasound probe and displaying a guideline indicating the insertion direction of a puncture needle based on the image of the puncture needle in the polymer gel portion so that it can be confirmed whether the puncture needle reaches a target site, that is, the puncture needle is correctly aimed at the target site.

### SUMMARY OF THE INVENTION

In paracentesis, thinner needles reduce patient burdens and invasiveness and therefore the smallest possible puncture needle is selected in consideration of the risk or the like. However, the thinner the needle is, the more the capability of imaging the needle on the ultrasound image is reduced. The needle is imaged discontinuously and the position or the shape of the puncture needle cannot be clearly displayed.

Since ultrasonic waves reflected on the puncture needle return toward the ultrasound probe at a smaller insertion angle of the puncture needle with respect to a subject, that is, at a larger angle formed between the puncture needle and a direction in which the ultrasound probe transmits the ultrasonic waves. Therefore, the puncture needle is displayed on the ultrasonic image at a higher definition and is more clearly seen.

Therefore, a doctor may insert a puncture needle from a position away from an ultrasound probe or shift the position of a target site in the image to reduce the insertion angle of the puncture needle so as to perform paracentesis in a state in which the puncture needle is more clearly seen on the ultrasound image.

There is a limit on the positional shift of the target site on the image and therefore the insertion angle cannot be significantly reduced. Accordingly, it is necessary to insert the puncture needle from a position away from the ultrasound probe so that the puncture needle may be inserted at a smaller insertion angle.

However, in the case of guiding the puncture needle in the groove formed on one lateral side of the ultrasound probe as described in JP 2010-115246 A, the puncture needle is to be brought into contact with the ultrasound probe and therefore the puncture needle cannot be guided along a predetermined insertion path if it is inserted at a position away from the ultrasound probe.

Also in the case of displaying the guidelines indicating the insertion direction of the puncture needle based on the image of the puncture needle in the polymer gel portion as in JP 2000-166918 A, the puncture needle in the polymer gel portion is to be displayed on the ultrasound image and therefore the guideline indicating the insertion direction of the puncture needle cannot be displayed if it is inserted at a position away from the ultrasound probe.

Accordingly, high levels of techniques and doctor's skills for paracentesis are necessary to insert a puncture needle from a position away from an ultrasound probe so that the puncture needle may correctly reach a target site.

An object of the present invention is to solve the foregoing prior art problems and to provide an ultrasound diagnostic apparatus which ensures that a puncture needle inserted from a position away from an ultrasound probe to reduce the insertion angle can also reach a target site.

In order to achieve the above object, the invention provides the ultrasound diagnostic apparatus comprising: an ultrasound probe which transmits ultrasonic waves toward a subject, receives ultrasonic echoes reflected on the subject and output reception signals; image production means for producing an ultrasound image of the subject based on the reception signals outputted from said ultrasound probe; image display means for displaying said ultrasound image generated by said image production means; position designating means for designating a target position where paracentesis is performed; insertion position calculating means for calculating an insertion position from information on the target position designated by said position designating means and a predetermined insertion angle; and insertion position display means for displaying the insertion position calculated by said insertion position calculating means.

In this case, it is preferred that said insertion position display means displays said insertion position by irradiation of the insertion position with light.

Further, it is preferred that said insertion position display means displays said insertion position by irradiation with a laser beam.

Alternatively, it is preferred that said insertion position display means displays said insertion position by showing on said image display means a distance value from said ultrasound probe to said insertion position.

Preferably, the ultrasound diagnostic apparatus further comprise a plate-like position guide member fixed to said ultrasound probe.

Alternatively, the ultrasound diagnostic apparatus further comprise a puncture guide having a plate-like position guide member fixed to said ultrasound probe and an angle guide member movably fixed to said position guide member and displaying an insertion angle for use in paracentesis, said insertion position display means displaying said insertion position by irradiation of said position guide member with light.

Further, it is preferred that said image display means displays said ultrasound image and guidelines obtained by calculating an insertion path of a puncture needle in the paracentesis from the information on the target position designated by said position designating means and the predetermined insertion angle.

Preferably, the ultrasound diagnostic apparatus further comprise angle adjustment means for changing said predetermined insertion angle.

Further, it is preferred that said predetermined insertion angle is appropriately determined in a range of 10° to 20°.

Further, it is preferred that said position designating means designates the target position on the ultrasound image displayed in said image display means according to an inputted instruction.

Alternatively, it is preferred that said position designating means analyzes said ultrasound image to calculate the target position.

The ultrasound diagnostic apparatus of the invention which is configured to include position designating means for designating a target position in paracentesis on an ultrasound image; insertion position calculating means for calculating an insertion position from information on the designated target position and a predetermined insertion angle and insertion position display means for displaying the insertion position calculated by the insertion position calculating means, ensures that a puncture needle inserted at a position away from an ultrasound probe can also reach a target site.

### BRIEF DESCRIPTION OF THE DRAWINGS

FIG. 1 is a conceptual diagram showing an embodiment of an ultrasound diagnostic apparatus of the invention.

FIG. 2A is a conceptual diagram showing an ultrasound probe that may be used in the ultrasound diagnostic apparatus shown in FIG. 1, FIG. 2B is a conceptual diagram showing the ultrasound probe shown in FIG. 2A, a puncture needle and a target site, and FIG. 2C is a conceptual diagram showing an angle guide member of the ultrasound probe shown in FIG. 2A.

FIG. 3 is a block diagram showing the configuration of the ultrasound diagnostic apparatus shown in FIG. 1.

FIGS. 4A to 4C each schematically show an ultrasound image for illustrating the operation of the ultrasound diagnostic apparatus shown in FIG. 1 when the insertion Position is calculated.

FIG. 5 is a schematic diagram of an ultrasound image on which guidelines for the puncture needle are shown.

FIG. 6 is a schematic diagram showing another example of the insertion position display means.

### DETAILED DESCRIPTION OF THE INVENTION

Next, the ultrasound diagnostic apparatus of the invention is described in detail by referring to the preferred embodiments shown in the accompanying drawings.

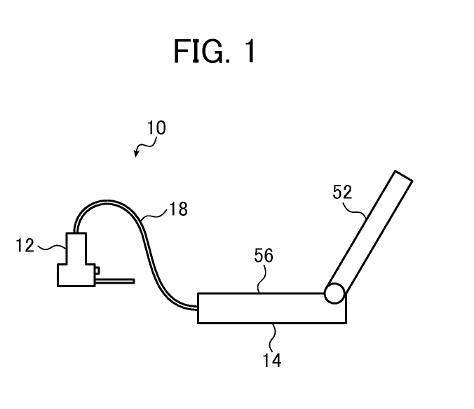

FIG. 1 is a conceptual diagram showing an embodiment of the ultrasound diagnostic apparatus of the invention.

The illustrated ultrasound diagnostic apparatus 10 is basically of a known type except that it is configured to calculate the insertion position from the target position inputted by reference to a generated ultrasound image and a preset insertion angle and to display the calculated insertion position.

The ultrasound diagnostic apparatus 10 of the invention includes an ultrasound probe 12, and a diagnostic apparatus body 14 which is connected to the ultrasound probe 12 via a communication cable 18.

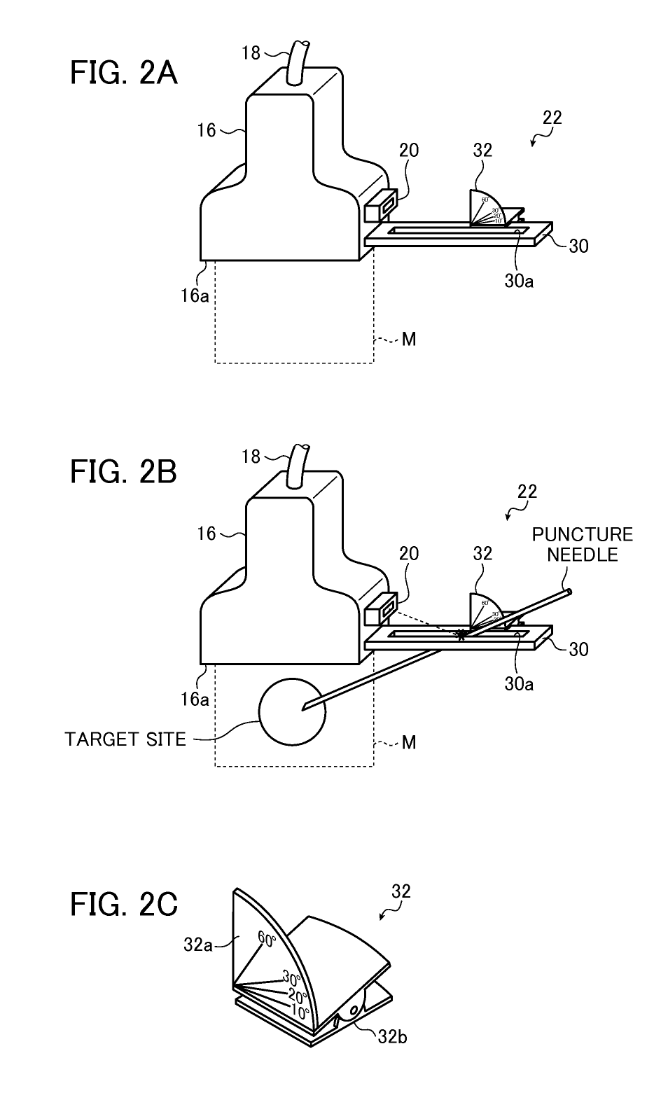

FIG. 2A is a schematic diagram showing the ultrasound probe 12. FIG. 2B is a schematic diagram showing the ultrasound probe 12, a puncture needle and a target site when paracentesis is performed.

The ultrasound probe 12 includes a probe body 16, the communication cable 18, an insertion position display means 20 and a puncture guide 22.

The probe body 16 is basically of a known type and may be of a linear scan type, convex scan type or sector scan type.

The probe body 16 has an ultrasound transmission/reception surface 16a for the transmission and the reception of ultrasonic waves. The insertion position display means 20 and the puncture guide 22 are disposed on one lateral side of the ultrasound transmission/reception surface 16a of the probe body 16 in the transverse direction of a scan surface M formed with ultrasonic waves transmitted from the ultrasound transmission/reception surface 16a.

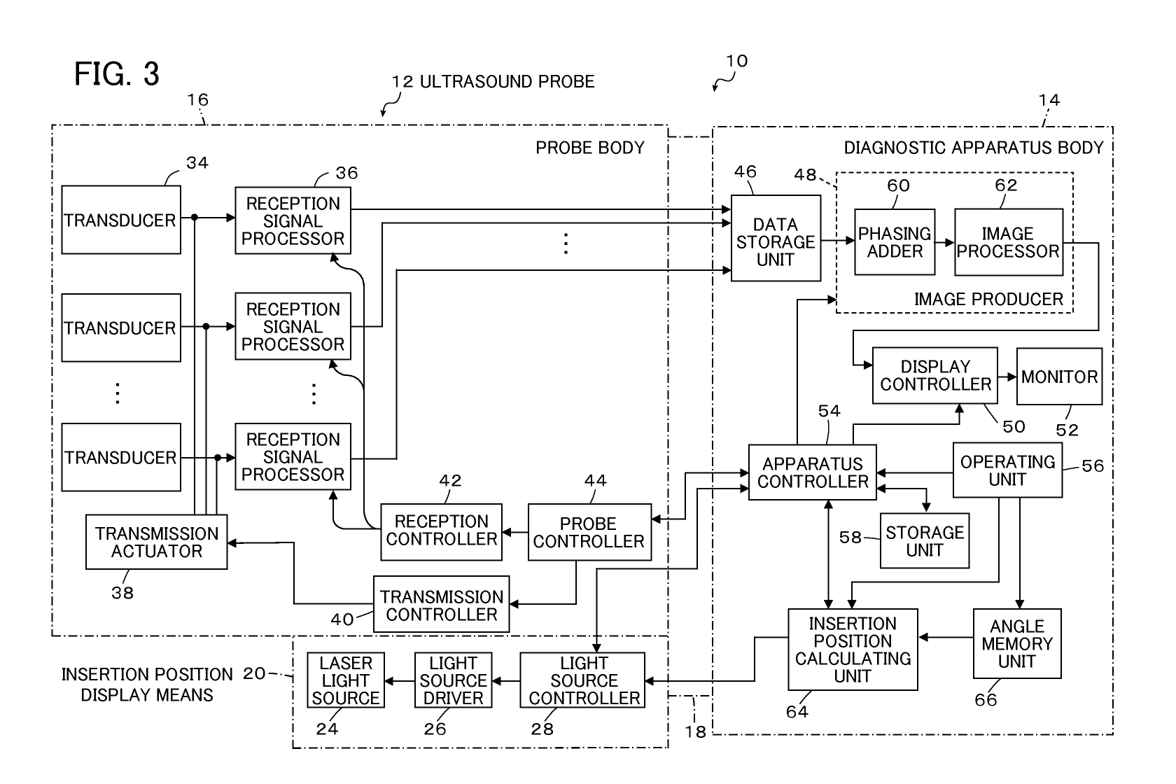

The insertion position display means 20 is disposed on the lateral side of the ultrasound transmission/reception surface 16a of the probe body 16 in the lateral direction of the scan surface M and a proper insertion position of the puncture needle is displayed by irradiation of the insertion position with a laser beam when paracentesis is performed using the ultrasound diagnostic apparatus 10. In the insertion position display means 20, a light source driver 26 causes a laser light source 24 for emitting laser beams to rotate about an axis perpendicular to the scan surface M to change the laser beam irradiation position to thereby irradiate the insertion position with a laser beam (see FIG. 3). This point will be described in detail later.

The insertion position at which the puncture needle can be inserted at a predetermined insertion angle is calculated in an insertion position calculating unit 64 of the diagnostic apparatus body 14 from a predetermined set insertion angle and a designated target position. This point will also be described in detail later.

The illustrated embodiment preferably includes the puncture guide 22 and therefore a laser beam from the insertion position display means 20 is irradiated on a position guide member 30 of the puncture guide 22 in the vicinity of the insertion position (see FIG. 2B).

The predetermined insertion angle is preferably an angle appropriately determined in a range of 10° to 20°. At an insertion angle within the foregoing range, ultrasonic waves reflected on the puncture needle return toward the ultrasound probe and therefore the puncture needle is displayed on the ultrasound image at a higher definition and is more clearly seen on the ultrasound image.

The insertion position display means 20 displays the insertion position calculated by the insertion position calculating unit 64 by irradiation with a laser beam as described above. The insertion of the puncture needle from the position at which the laser beam is irradiated ensures that the puncture needle reaches the target site even in cases where a doctor inserts the puncture needle from a position away from the ultrasound probe in paracentesis for the insertion at a predetermined small insertion angle.

In the preferred embodiment, the illustrated ultrasound diagnostic apparatus 10 includes the puncture guide 22.

The puncture guide 22 has the position guide member 30 for guiding the insertion position and an angle guide member 32 for guiding the insertion angle which are used in paracentesis.

The position guide member 30 is an elongated plate-like member fixed to the probe body 16 so that its largest surface is orthogonal to the scan surface M, and has a through slot 30a formed in the center in the width direction or lateral direction of the member 30 so that the extension of the scan surface M passes through the slot 30a or coincides with one of wall surfaces thereof.

The through slot 30a is preferably formed so that the extension of the scan surface M coincides with one of walls in the direction in which the through slot 30a extends or passes through the center of the through slot 30a.

When the insertion position display means 20 displays the insertion position, the laser beam from the insertion position display means 20 is irradiated on the surface of the position guide member 30 to display the insertion position.

By providing the position guide member 30 on the lateral side of the probe body 16 so that the laser beam is irradiated on the position guide member 30 to display the insertion position, the displayed insertion position may be proper even when a subject has a curved surface.

The through slot 30a of the position guide member 30 through which the puncture needle is inserted in paracentesis may serve as the positional guide in the direction parallel to the scan surface M which enables the insertion path of the puncture needle to be flush with the scan surface M in the direction perpendicular to the scan surface M.

The method of fixing the position guide member 30 to the probe body 16 is not particularly limited and various known fixing methods such as fixation with screws may be used. Alternatively, the position guide member 30 may be integrally formed with a housing of the probe body 16.

FIG. 2C is a schematic diagram showing the angle guide member 32 of the ultrasound probe 12 shown in FIG. 2A.

The angle guide member 32 is attached to the position guide member 30 so as to be movable in its longitudinal direction and has an angle display plate 32a on which the angle with respect to the surface of the position guide member 30 is shown.

In the illustrated embodiment, the angle guide member 32 has a clip portion 32b for fixing to the position guide member 30, and the clip portion 32b enables the angle guide member 32 to be movably attached to the position guide member 30. The angle guide member 32 is attached to the position guide member 30 so that the angle display plate 32a may be parallel to the scan surface M.

When the insertion position display means 20 irradiates a laser beam on the position guide member 30 to display an insertion position, the angle guide member 32 is fixed to the position guide member 30 at the insertion position. The angle display plate 32a displays an insertion angle for use in inserting a puncture needle from the displayed insertion position.

The angle display plate 32a is marked with angles such as 10°, 20°, 30° and 60° as in the illustrated case. The angle display plate 32a is preferably marked with at least one angle set by default.

According to such a configuration that the puncture guide includes the angle guide member 32 movably attached to the position guide member 30, the puncture needle can be easily inserted in paracentesis at a predetermined insertion angle irrespective of doctor's skills by fixing the angle guide member 32 to the position guide member 30 at the insertion position displayed on the position guide member 30 by irradiation with a laser beam from the insertion position display means 20 and inserting the puncture needle at the insertion angle indicated by an angle shown on the angle display plate 32a.

In the illustrated case, the angle guide member 32 is fixed to the position guide member 30 with a clip portion 32b but the fixation method is not limited to this. For example, various known methods may be used as exemplified by fixing it with screws and fixing it slidably along the plane parallel to the scan surface M.

In the illustrated case, the angle guide member 32 has the angle display plate 32a marked with an angular scale. However, this is not the sole case of the invention and the angle guide member 32 may have a groove for guiding a puncture needle at the same angle as the predetermined set angle.

Next, the internal configuration of the ultrasound diagnostic apparatus 10 is shown in FIG. 3.

The probe body 16 includes a plurality of ultrasound transducers 34 making up a one-dimensional or two-dimensional oscillator array, and the transducers 34 are connected to their corresponding reception signal processors 36. The transducers 34 are connected to a transmission controller 40 via a transmission actuator 38, and the reception signal processors 36 are connected to a reception controller 42. The transmission controller 40 and the reception controller 42 are connected to a probe controller 44.

The reception signal processors 36 and the probe controller 44 are connected via the communication cable to a data storage unit 46 and an apparatus controller 54 of the diagnostic apparatus body 14, respectively.

The transducers 34 each transmit ultrasonic waves according to actuation signals supplied from the transmission actuator 38 and receive ultrasonic echoes from the subject to output reception signals. Each of the transducers 34 is composed of an oscillator including, for example, a piezoelectric body made of a piezoelectric ceramic typified by PZT (lead zirconate titanate) or a piezoelectric polymer typified by PVDF (polyvinylidene fluoride) and an electrode provided on each end of the piezoelectric body.

When the electrodes of each of the oscillators are supplied with a pulsed voltage or a continuous-wave voltage, the piezoelectric body expands and contracts to cause the oscillator to generate pulsed or continuous ultrasonic waves. These ultrasonic waves are synthesized to form an ultrasonic beam. Upon reception of propagating ultrasonic waves, each oscillator expands and contracts to generate an electric signal, which is then outputted as an ultrasonic reception signal.

The transmission actuator 38 includes, for example, a plurality of pulsers and adjusts the delay amounts of actuation signals for the respective transducers 34 based on a transmission delay pattern selected by the transmission controller 40 so that the ultrasonic waves transmitted from the transducers 34 form a broad ultrasonic beam covering an area of a tissue of the subject and supplies the transducers 34 with the adjusted actuation signals.

Under the control of the reception controller 42, the reception signal processor 36 for each channel subjects the reception signal outputted from the corresponding transducer 34 to quadrature detection or quadrature sampling to produce a complex baseband signal and samples the complex baseband signal to generate sample data containing information on the area of the tissue. The reception signal processors 36 may generate sample data by performing data compression for highly efficient coding on the data obtained by sampling the complex baseband signals.

The probe controller 44 controls various components of the probe body 16 according to various control signals transmitted from the diagnostic apparatus body 14.

The diagnostic apparatus body 14 includes the data storage unit 46, which is connected to an image producer 48. The image producer 48 is connected to a monitor 52 via a display controller 50. The apparatus controller 54 is connected to the image producer 48 and the display controller 50. The apparatus controller 54 is further connected to an operating unit 56 for an operator to perform input operations, a storage unit 58 for storing operation programs, and the insertion position calculating unit 64 for calculating the insertion position. The insertion position calculating unit 64 is connected to an angle memory unit 66 for storing set insertion angles.

The operating unit 56 sets imaging menus and imaging conditions and issues instructions for imaging of a subject. The operating unit 56 is provided with input means such as an enter key, a dial button, a trackball and a touch panel for setting imaging menus, imaging conditions and the like.

The ultrasound diagnostic apparatus 10 of the invention calculates the insertion position from the target position (target site position) inputted and set by reference to a generated ultrasound image and the preset insertion angle and displays the calculated insertion position.

The operating unit 56 also has a function of inputting an instruction for calculating/displaying the insertion position. The operating unit 56 further has a function of inputting instructions for inputting/setting the target position and setting the insertion angle.

The operating unit 56 supplies to the apparatus controller 54 the instruction inputted for calculating/displaying the insertion position, the instruction inputted for setting the target position, and the instruction inputted for setting the insertion angle.

The data storage unit 46 is constituted by a memory, a hard disk, or the like and stores at least one frame of sample data transmitted from the reception signal processors 36 of the ultrasound probe 12 via the communication cable 18.

The image producer 48 performs reception focusing on each frame of sample data read out from the data storage unit 46 to generate an image signal representing an ultrasound diagnostic image. The image producer 48 includes a phasing adder 60 and an image processor 62.

The phasing adder 60 selects one reception delay pattern from a plurality of previously stored reception delay patterns according to the reception direction set in the apparatus controller 54 and, based on the selected reception delay pattern, provides the complex baseband signals represented by the sample data with respective delays and adds them to perform the reception focusing. By this reception focusing, a baseband signal (sound ray signal) where the ultrasonic echoes are well focused is generated.

The image processor 62 generates a B-mode image signal, which is tomographic image information on a tissue inside the subject, according to the sound ray signal generated by the phasing adder 60. The image processor 62 includes an STC (sensitivity time control) part and a DSC (digital scan converter). The STC part corrects the sound ray signal for the attenuation due to distance according to the depth to the reflection position of the ultrasonic waves. The DSC converts the sound ray signal corrected by the STC part into an image signal compatible with an ordinary television signal scanning mode (performs raster conversion) and performs required image processing such as gradation processing to generate a B mode image signal.

The display controller 50 causes the monitor 52 to display an ultrasound diagnostic image according to the image signal generated by the image producer 48. The monitor 52 includes a display device such as an LCD, for example, and displays an ultrasound diagnostic image under the control of the display controller 50.

The angle memory unit 66 is a unit for storing insertion angle values that may be used to calculate the insertion position in the insertion position calculating unit 64.

The insertion angle value to be stored in the angle memory unit 66 may be a preset value, an angle appropriately determined in a range of 10° to 20° or a value inputted from the operating unit 56.

The insertion position calculating unit 64 calculates the insertion position from the target position information inputted from the operating unit 56 and the insertion angle value stored in the angle memory unit 66. The distance x between the target and the insertion position can be determined from the formula: x=d/tan θ (where d is the depth from the probe body 16 to the target and θ is the insertion angle).

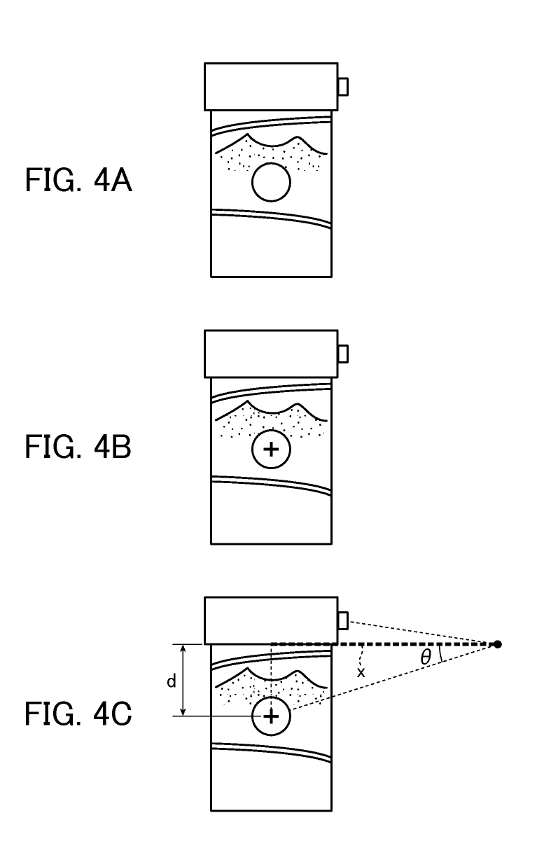

The operation of the insertion position calculating unit 64 in calculating the insertion position is described below in detail with reference to FIGS. 4A to 4C schematically showing ultrasound images.

FIG. 4A is a schematic diagram of a generated ultrasound image. The position of the probe body 16 corresponding to the ultrasound image is shown for the sake of convenience.

As shown in FIG. 4A, the generated ultrasound image is a cross-sectional image located under the probe body 16.

When an instruction for displaying the insertion position is inputted in the operating unit 56, a cursor appears on the ultrasound image displayed on the monitor 52 and a screen for making an operator to point the cursor to a target or target site is displayed.

The operator uses a trackball or the like in the operating unit 56 to point the cursor to the target on the ultrasound image and inputs an instruction for designating the position of the target (FIG. 4B).

Upon the designation of the target position, the target position information is supplied to the insertion position calculating unit 64. From the supplied target position information (target depth d) and the insertion angle θ read out from the angle memory unit 66, the insertion position calculating unit 64 calculates the distance x between the probe body 16 and the insertion position (FIG. 4C).

The insertion position calculating unit 64 supplies the calculated distance x to the insertion position to a light source controller 28.

The apparatus controller 54 controls the units in the diagnostic apparatus body 14. The apparatus controller 54 is connected to the probe controller 44 of the probe body 16 via the communication cable 18 and supplies control signals for controlling the operation of the probe body 16 to the probe controller 44. The apparatus controller 54 is connected to the light source controller 28 of the insertion position display means 20 via the communication cable 18 and supplies control signals for controlling the operation of the insertion position display means 20 to the light source controller 28.

In such a diagnostic apparatus body 14, while the image producer 48, the display controller 50, the apparatus controller 54, and the insertion position calculating unit 64 are each constituted by a CPU and an operation program for causing the CPU to perform various kinds of processing, they may be constituted by a digital circuit. The aforementioned operation program is stored in the storage unit 58.

The insertion position display means 20 includes the laser light source 24 for emitting laser beams, the light source driver 26 in which a drive source such as a motor is used to rotate the laser light source 24 about the axis perpendicular to the scan surface M to modify the laser beam irradiation position thereby showing the insertion position with a laser beam, and the light source controller 28 for controlling the light source driver 26.

In the case of using the position guide member 30 as in the illustrated case, the light source driver 26 preferably rotates the laser light source 24 so that the laser beam may move on the position guide member 30 preferably in close proximity to the through slot 30a parallel to the extension of the scan surface M. As will be described later, when the position guide member 30 is not used, the light source driver 26 preferably rotates the laser light source so that the laser beam may move on the extension of the scan surface M.

The light source controller 28 is connected via the communication cable 18 to the apparatus controller 54 and the insertion position calculating unit 64 of the diagnostic apparatus body 14. The light source controller 28 acquires the insertion position calculated in the insertion position calculating unit 64 and controls the light source driver 26 so that the laser beam from the laser light source 24 may be irradiated on the position guide member 30 at the insertion position. The light source controller 28 controls ON/OFF of the laser light source 24 based on the instruction from the apparatus controller 54.

Next, the operation of the ultrasound diagnostic apparatus 10 is described.

First, a common ultrasound examination is made. An operator brings the ultrasound transmission/reception surface 16a of the ultrasound probe 12 into contact with the surface of a subject. In such a state, the transducers 34 transmit ultrasonic waves according to the actuation signals supplied from the transmission actuator 38 of the probe body 16, and the reception signals outputted from the transducers 34 that have received the ultrasonic echoes from the subject are supplied to the corresponding reception signal processors 36 to generate sample data, which are then transmitted via the communication cable 18 to the diagnostic apparatus body 14 and stored in the data storage unit 46. Further, the sample data is read out from the data storage unit 46 frame by frame, and the image producer 48 generates the image signal and, based on this image signal, the display controller 50 causes the monitor 52 to display the ultrasound diagnostic image.

When an instruction for displaying the insertion position is inputted in the operating unit 56 in order to perform paracentesis using the displayed ultrasonic image, a cursor appears on the ultrasound image displayed in the monitor 52 and a screen for making the operator to point the cursor to a target is displayed.

When the operator designates the target position from the operating unit 56, the target position information is supplied to the insertion position calculating unit 64. The insertion position calculating unit 64 calculates the insertion position from the target position information and the insertion angle read out from the angle memory unit 66 and supplies the insertion position information to the light source controller 28 of the insertion position display means 20.

The light source controller 28 of the insertion position display means 20 controls the light source driver 26 based on the supplied insertion position information to cause the laser light source 24 to rotate so that a proper insertion position at which the puncture needle is inserted at a predetermined insertion angle is displayed by irradiation of the insertion position with the laser beam from the laser light source.

Since the insertion position display means 20 displays the insertion position at which the puncture needle is inserted at the predetermined insertion angle as described above, insertion of the puncture needle from the position at which the laser beam is irradiated ensures that the puncture needle can reach the target site even in cases where a doctor inserts the puncture needle from a position away from the ultrasound probe in paracentesis for the insertion at a predetermined small insertion angle.

The present invention is basically as described above.

In the illustrated case, the calculated insertion position is only displayed using the insertion position display means 20 including the laser light source but this is not the sole case of the invention.

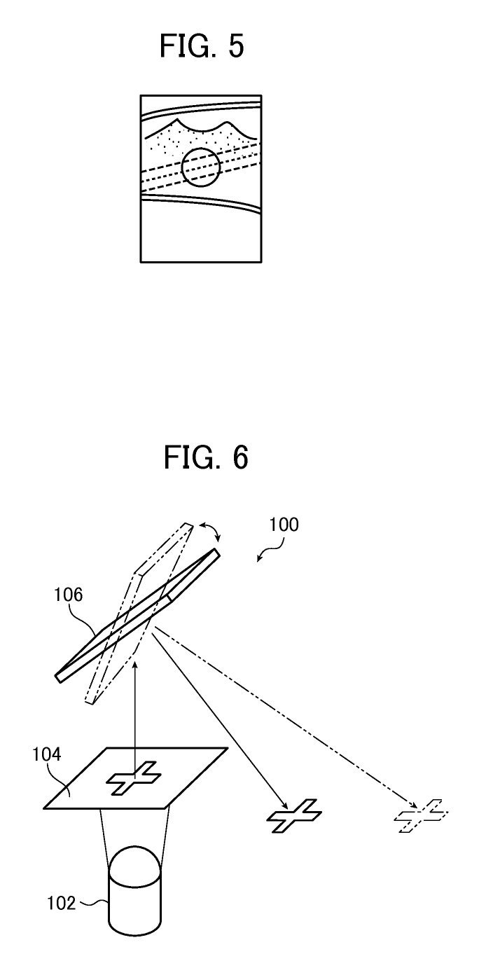

For example, the insertion oath of the puncture needle may be calculated from the insertion position information calculated in the insertion position calculating unit 64 and the predetermined insertion angle stored in the angle memory unit 66 and be displayed as guidelines on the ultrasound image displayed in the monitor 52 as in the schematic diagram of the ultrasound image shown in FIG. 5.

There are also cases where a doctor performs paracentesis from a direction opposite from the direction on the ultrasound image with respect to the ultrasound probe 12. Therefore, left and right of the ultrasound image may be reversed according to the instruction from the operating unit 56. Left and right may also be reversed for the guidelines of the puncture needle if they are displayed.

The illustrated ultrasound diagnostic apparatus 10 is configured such that an operator manipulates the operating unit 56 to point the cursor to a target site position on the ultrasound image and designate the target position to thereby acquire the target position information. However, this is not the sole case of the invention and the ultrasound diagnostic apparatus may be, for example, configured to analyze the ultrasound image and calculate the position of a target such as a tumor. In such a case, the ultrasound diagnostic apparatus is preferably configured such that the operator can correct as necessary the target position from the operating unit.

The illustrated ultrasound diagnostic apparatus 10 is configured such that the ultrasound probe 12 includes the puncture guide 22 and a laser beam from the insertion position display means 20 is irradiated on the surface of the puncture guide 22 (more specifically the position guide member 30). However, this is not the sole case of the invention. For example, the ultrasound diagnostic apparatus 10 may be configured such that a laser beam from the insertion position display means 20 is irradiated on the skin surface of a subject instead of using the puncture guide 22.

The method of displaying the insertion position using the insertion position display means 20 is not limited to one in which the insertion position is displayed by irradiation with a laser beam, and the insertion position may be displayed using a light source other than a laser such as an LED.

FIG. 6 is a schematic diagram showing another example of the insertion position display means that may be used in the ultrasound diagnostic apparatus of the invention.

An insertion position display means 100 shown in FIG. 6 includes a light source 102, a slit member 104 and a reflector 106.

The light source 102 is of a known type such as LED. The slit member 104 and the reflector 106 are disposed downward in the direction of light travel from the light source 102 and the light source 102 emits light toward the slit member 104 and the reflector 106.

The slit member 104 is a plate-like member having a cross-shaped light-transmitting window formed in the center thereof and the light-transmitting window passes part of light from the light source 102. Only part of light irradiated on the slit member 104 which corresponds to the shape of the light-transmitting window impinges on the reflector 106.

The reflector 106 is used to reflect light which was emitted from the light source 102 and passed through the slit member 104 to irradiate the insertion position.

The reflector 106 is mounted on the axis perpendicular to the scan surface M so as to be rotatable by a drive source such as motor (not shown). The reflector 106 rotates based on the calculation result of the insertion position in the insertion position calculating unit 64 to irradiate the insertion position with light.

As described above, a light source other than a laser (e.g., LED) may also be used, and particularly in the case of using a nondirectional light source, a slit member and a reflector may be used as in the insertion position display means 100 shown in FIG. 6 to irradiate the insertion position with light.

The method of displaying the insertion position is not limited to a method of irradiating the insertion position with light (laser beam) and the distance value from the probe body to the insertion position may be displayed on the monitor 52. In such a case, the surface of the position guide member 30 in the puncture guide is preferably marked with a distance scale.

The illustrated ultrasound diagnostic apparatus 10 is configured such that a wired connection is established between the probe body 16 and the diagnostic apparatus body 14 via the communication cable 18 to perform the transmission and reception of various data. However, this is not the sole case of the invention and wireless transmission and reception of data may be performed between the probe body 16 and the diagnostic apparatus body 14.

While the ultrasound diagnostic apparatus of the invention has been described above in detail, the invention is by no means limited to the above embodiments, and various improvements or modifications may be made without departing from the scope and spirit of the present invention.

What is claimed is:

1. An ultrasound diagnostic apparatus comprising:
an ultrasound probe which transmits ultrasonic waves toward a subject, receives ultrasonic echoes reflected on the subject and output reception signals;
image production means for producing an ultrasound image of the subject based on the reception signals outputted from said ultrasound probe;
image display means for displaying said ultrasound image generated by said image production means;
position designating means for designating a target position where paracentesis is performed;
insertion position calculating means for calculating an insertion position from information on the target position designated by said position designating means and a predetermined insertion angle; and
insertion position display means for displaying the insertion position calculated by said insertion position calculating means.

2. The ultrasound diagnostic apparatus according to claim 1, wherein said insertion position display means displays said insertion position by irradiation of the insertion position with light.

3. The ultrasound diagnostic apparatus according to claim 2, wherein said insertion position display means displays said insertion position by irradiation with a laser beam.

4. The ultrasound diagnostic apparatus according to claim 1, wherein said insertion position display means displays said insertion position by showing on said image display means a distance value from said ultrasound probe to said insertion position.

5. The ultrasound diagnostic apparatus according to claim 1, further comprising a position guide member fixed to said ultrasound probe.

6. The ultrasound diagnostic apparatus according to claim 2, which further comprises a puncture guide having a position guide member fixed to said ultrasound probe and an angle guide member movably fixed to said position guide member and displaying an insertion angle for use in paracentesis, said insertion position display means displaying said insertion position by irradiation of said position guide member with light.

7. The ultrasound diagnostic apparatus according to claim 1, wherein said image display means displays said ultrasound image and guidelines obtained by calculating an insertion path of a puncture needle in the paracentesis from the information on the target position designated by said position designating means and the predetermined insertion angle.

8. The ultrasound diagnostic apparatus according to claim 1, further comprising angle adjustment means for changing said predetermined insertion angle.

9. The ultrasound diagnostic apparatus according to claim 1, wherein said predetermined insertion angle is appropriately determined in a range of 10° to 20°.

10. The ultrasound diagnostic apparatus according to claim 1, wherein said position designating means designates the target position on the ultrasound image displayed in said image display means according to an inputted instruction.

11. The ultrasound diagnostic apparatus according to claim 1, wherein said position designating means analyzes said ultrasound image to calculate the target position.
